(19) United States
(12) Patent Application Publication
(10) Pub. No.: US 2002/0111661 A1
(43) Pub. Date: Aug. 15, 2002

(54) Multiple electrode lead body for spinal cord stimulation

(75) Inventors: Thomas E. Cross, St. Francis, MN (US); Gabor R. Racz, Lubback, TX (US)

Correspondence Address: BANNER & WITCOFF, LTD., TEN SOUTH WACKER DRIVE, SUITE 3000, CHICAGO, IL, 60606, US

(73) Assignee: Medtronic, Inc., Minneapolis, MN

(21) Appl. No.: 10/025,112

(22) Filed: Dec. 19, 2001

Related U.S. Application Data
(63) Continuation-in-part of application No. 09/070,052, filed on Apr. 30, 1998, now abandoned.

(51) Int. Cl. A61N 1/05

(52) U.S. Cl. 607/117

(57) ABSTRACT

An implantable medical lead for spinal cord stimulation includes a lead paddle having an array of multiple electrode contacts each coupled to a wire conductor of a lead body. The wire conductor in turn may be coupled to an implantable pulse generator or other stimulation device. The lead paddle with an array of electrode contacts provides more complete electrical stimulation coverage to targeted human tissue because there is no potential for a targeted fiber to pass through the array of electrode contacts without having some potential for correct electrical stimulation by a contact.

This application claims priority to U.S. application Ser. No. 09/070,052, filed Apr. 30,1998.

### FIELD OF THE INVENTION

The present invention relates generally to implantable medical stimulators and more particularly to medical leads having a plurality of electrode contacts.

### BACKGROUND OF THE INVENTION

Electrical stimulation of electrically excitable tissue such as the brain and/or nerve tissue of the spinal cord or peripheral nerve can result in pain reduction and/or elimination for the living organism having the stimulated electrically excitable tissue.

Thus, for example, medical leads having electrode contacts have been implanted near the spinal column of the human body to provide pain relief for chronic intractable pain. The nerve tissue within the spinal column is stimulated electrically to reduce pain sensations at other parts of the body.

Depending on the location of the pain sensation, and the particularities of each different human body, the parameters of the stimulation signals applied near the electrically excitable tissue are adjusted to optimize pain reduction or elimination.

For example, the area of excitation within the spinal column and the intensity of excitation can be varied by corresponding adjustment of the parameters of the stimulation signals.

In order to vary the area of excitation, an array of electrodes may be implanted near the nerve tissue within the spinal column or peripheral nerve. Then, each of those electrodes can be configured to have a respective one of a positive, negative, or neutral polarity such that the desired area of the nerve tissue within the spinal column is electrically stimulated. In addition, parameters of the respective stimulation signal applied on each of those implanted electrodes can be varied for a corresponding variation in area of excitation within the spinal column and in the intensity of excitation at the pain site. Once the array of electrodes is implanted, a clinician who is knowledgeable of the effects of electrical stimulation may vary the parameters of the respective stimulation signal applied on each of the implanted electrodes. The patient may rate the effectiveness in pain reduction for each variation in the parameters of the stimulation signals. If electrical stimulation of nerve tissue does result in sufficient pain reduction for the patient, then the medical lead is implanted for the long term with stimulation signals having parameters that lead to optimized pain reduction for the particular patient.

However, prior art electrode arrays do not provide adequate stimulation coverage. In some spinal cord stimulation cases, the best stimulation points, for example, nerve fibers, are sufficiently far apart that the side-to-side spacing between electrodes on current leads is not adequate to span them. In addition, the electrode arrays have electrode contacts that are spaced axially and longitudinally along a lead body such that gaps remain between adjacent contacts. As a result of these gaps, nerve fibers may pass between the electrode contacts and be unavailable for stimulation. This is highly undesirable when a clinician is trying to cover an entire targeted area because the clinician does not necessarily know where the desired fibers are within this targeted area of excitation.

### BRIEF SUMMARY OF THE INVENTION

The present invention recognizes and provides a solution to the problems of inadequate electrode array coverage in providing a unique lead paddle that has an array of spaced apart, but yet stimulation overlapping electrode contacts for complete nerve fiber stimulation of a targeted area.

Accordingly, an object of the present invention is to provide for a unique implantable medical lead having a lead paddle which includes a plurality of electrode contacts, such as an array, for transmitting stimulation signals to surrounding human tissue. Another object of the invention is to provide a lead paddle that has an array of stimulation overlapping electrode contacts for more complete stimulation coverage of a targeted area of excitation. Further, another object of the invention is to provide a lead paddle that has an array of electrode contacts that span distant nerve fibers and at the same time provide combinations that cover nerve fibers that may be close together. Yet another object of the invention is to provide a lead paddle that has an array of electrode contacts wherein the parameters of the stimulation signals applied to each electrode can be controlled to stimulate targeted fibers. Still another object of the invention is to provide a multiple electrode lead paddle that is curved laterally to approximately match the curve of the outside of the dura mater which encircles the spinal cord.

The present invention provides an implantable medical lead, for spinal cord stimulation, comprising a lead paddle having an array of about eight axially and laterally spaced electrode contacts. The array of electrode contacts provides for more complete coverage of targeted stimulation areas because the array of the present invention prevents nerve fibers from passing through the array without having some potential for correct electrical stimulation by a pair of electrode contacts.

The disclosed distribution of electrode contacts on a curved lead paddle also provides horizontal stimulation near the entry zone where the nerve enters the spinal cord.

This new arrangement of electrode contacts is based on a more current understanding of the pathological processes that are occurring horizontally in the spinal cord rather than just the previously understood vertical pathological changes going toward the brain. In order to stimulate near the entry zone where the nerve enters the spinal cord, the curved paddle with a lateral most contact point will allow lateral stimulation while the other electrode contact points on the curved paddle will allow stimulation axially along the spinal cord for pain relief. Additionally, the curved paddle allows for a more close proximity of the stimulating electrode to the spinal cord which reduces the voltage requirements because of the shorter distance between the stimulating electrode contact and the spinal cord. Moreover, the curved lead paddle ensures that horizontal migration of the lead paddle will not take place, unlike the conventional percutaneously placed electrodes.

The full range of objects, aspects and advantages of the invention are only appreciated by a full reading of this specification and a full understanding of the invention. Therefore, to complete this specification, a detailed description of the invention and the preferred embodiment follows, after a brief description of the drawing.

### BRIEF DESCRIPTION OF THE DRAWINGS

The invention will be described in relation to the accompanying drawings. In the drawings, the following figures have the following general nature:

FIG. 1 is a plan view of a medical lead having a lead paddle of the present invention coupled to lead bodies.

FIG. 2 is a plan view of the lead paddle having the preferred array of electrode contacts of the present invention.

FIG. 3 is a cross-section view of the lead paddle of FIG. 2 taken at line 3-3.

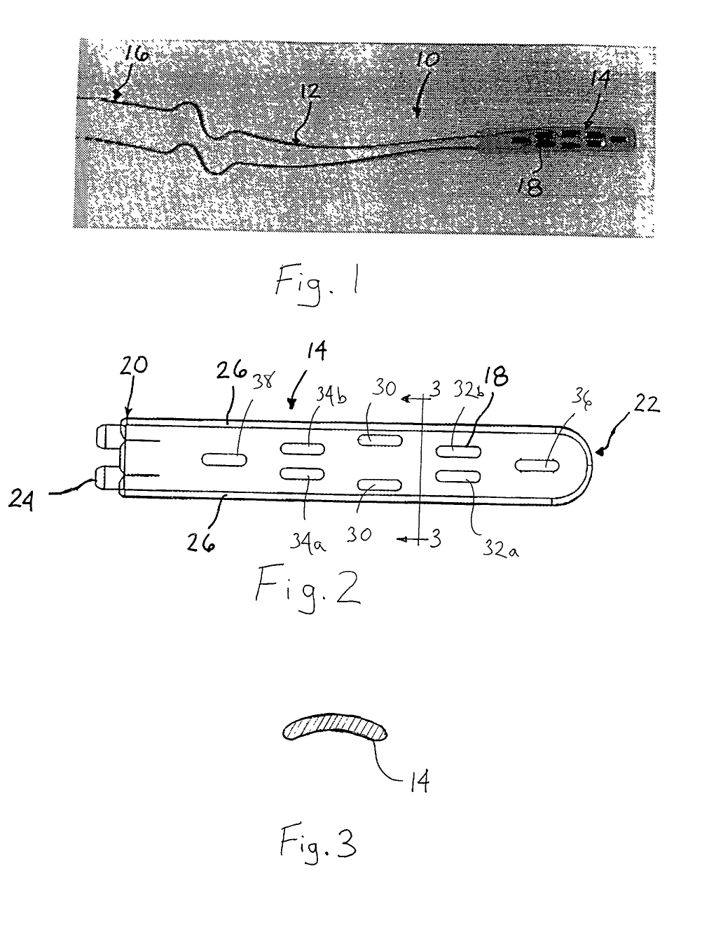

In the accompanying drawings, like reference numbers are used throughout the various figures for identical structures.

### DETAILED DESCRIPTION OF THE INVENTION

Referring to FIG .1, an exemplary embodiment of the medical lead 10 for spinal cord, peripheral nerve and deep brain stem stimulation comprises at least one lead body 12 and a preferred lead paddle 14. The lead paddle 14 has an array of electrode contacts 18 and is coupled at one end to the lead body. The lead body 12 further comprises at least one wire conductor. The number of wire conductors may be increased to two, three, or more, dependent on need and significant generally to the number of electrical signals to be generated.

Each proximal end 16 of a lead body such as the illustrated lead bodies 12 may be coupled to an implantable neurological pulse generator, additional, intermediate wiring, or other stimulation device. An example of such a neurological pulse generator is the ITREL II system from Medtronic, Inc., Minneapolis, Minn. The stimulation pulses produced by the implantable neurological pulse generator are carried from the pulse generator through the proximal ends 16 of the lead bodies 12, via the wire conductor, to distal ends of the lead bodies 12, and thereby to a coupled lead paddle 14 having at least one electrode contact 18.

One or more of the electrode contacts 18 on the lead paddle 14 transmit the stimulation pulses to targeted human tissue. As preferred, the illustrated structure transmits stimulation pulses from a pair of the contacts 18. The pair is selected through testing of the efficacy of alternate electrode pairs. Alternatively, the illustrated structure may transmit stimulation pulses from one electrode contact 18 or a plurality of electrode contacts 18 depending on the desired stimulation.

Though the preferred embodiment employs fully implantable elements, systems employing partially implanted generators and R-F coupling may also be used in the practice of the present invention. Such systems are also available from Medtronic, Inc., under the trademarks X-trel and Mattrix.

Each lead body 12 is generally a straight wire metal conductor within an insulating sheath. The insulating sheath is formed of an inert material such as polyurethane. Varieties of lead bodies are contemplated. Explanation of the reasoning for specific lead bodies is beyond this invention.

The lead paddle 14 of the present invention has a plurality of electrode contacts 18 arrayed along the length and across the width of the lead paddle 14. Varieties of alternate arrays and numbers of electrodes are contemplated. The lead paddle 14 with the array of electrode contacts 18 transmit stimulation signals to surrounding human tissue. The implantable pulse generator provides respective stimulation signals having specified signal parameters to selected contacts 18 in the array. Thus, depending on the desired location and amount of tissue stimulation, the parameters of the stimulation signals can be controlled and directed to selected electrode contacts for targeted stimulation. For spinal cord stimulation, the lead paddle 14 is placed outside the dura mater and stimulation occurs through the dura mater to the targeted tissue fibers. The lead paddle 14 is properly positioned, as known as a result of fluoroscopy and trial stimulation of tissue fibers.

Referring to FIG. 2, as most preferred, the lead paddle 14 has an array of eight electrode contacts 18 spaced axially along the length of the lead paddle and laterally across the width. This array of electrode contacts 18 spans distant stimulation points, for example, nerve fibers, and at the same time provides combinations that cover stimulation points that may be close together. Because the epidural space restricts the width of any implanted body, the array of the present invention must span distant stimulation points to maximize the number of nerve fibers that are stimulated through the array. A clinician may direct stimulation to various combinations of stimulation points covered by the array of the present invention by controlling the amount and frequency to each electrode contact 18.

Experience shows that beneficial stimulation occurs when the electrode contacts 18 are within 3 millimeters from the midline of the lead paddle 14. Thus, total lateral spacing of the electrode contacts 18 along the width of lead paddle 14 is preferably 6 millimeters. Variations of the spacing of the electrode contacts 18 and the width of the lead paddle 14 can be made and are contemplated. The axial length of the lead paddle 14 may be any suitable length to fit the desired number of electrode contacts 18 onto a lead paddle 14. The thickness of the lead paddle is sufficient to fit an electrode contact and accompanying wire conductor. It is preferred that the lead paddle 14 be as thin as possible to reduce the possibility of compression of the spinal cord.

In a preferred embodiment, and based on past studies to avoid lesions from smaller contact areas, the size of the electrode contacts 18 are approximately 12 square millimeters. However, electrode contacts of other suitable sizes are contemplated and within the scope of this invention. Thus, the lead paddle 14 has the shape of a slender elongated paddle.

The lead paddle 14 may be made of any suitable material, such as silicone rubber, adapted to be disposed within the human body. The lead paddle has a proximal end 20 and a distal end 22. The proximal end 20 provides at least one opening 24 for the wire conductors to pass into the lead paddle and couple to the electrode contacts 18. The distal end 22 is rounded and curved to prevent abrasion of human tissue for safer placement of the lead paddle at the desired stimulation area. The sides 26 of the lead paddle 14 are also rounded to prevent abrasion of tissue during implantation and while implanted. The lead paddle 14 may also be curved laterally to match the curvature of the dura mater, which encircles the spinal cord. A curved lead paddle 14 enhances the likelihood of fiber stimulation by allowing the electrode contacts 18 to be in close proximity to the targeted tissue fibers thus improving fiber recruitment. Moreover, a curved lead paddle 14 reduces the potential for compression of the spinal cord.

As most preferred, the electrode contacts 18 in the lateral direction provide stimulation overlap. As a result, there is no potential for targeted ascending and descending nerve fibers to pass through the array of electrode contacts 18 without having some potential for correct electrical stimulation by a contact.

In an exemplary embodiment of an array of eight electrode contacts, the array defines a diamond configuration. The diamond configuration allows for stimulation overlapping across the width of the lead paddle and adequate axial spacing. The diamond configuration also maximizes stimulation coverage across the paddle with a minimum number of electrodes. As depicted, a first pair of electrodes 30 are centrally located on the lead paddle and positioned near the periphery of the paddle. The first pair of electrodes defines a first pair width, which is the distance between the electrodes 30 of the first pair. As shown, there are no electrodes positioned within the width of the first pair of electrodes, only axially along the lead paddle. Also positioned on the lead paddle are second pairs 32a, 32b, and third pairs 34a, 34b of electrodes. These electrode pairs are positioned on both sides of the first pair 30 of electrodes between the paddle distal end 22 and proximal end 20, respectively. The second and third pairs of electrodes each have a pair width that is less than the width of the first pair. As with the first pair of electrodes, there are no electrodes positioned between the widths of the first and second pairs, only axially along the lead paddle. As depicted, there is a single electrode 36 positioned near the distal end 22 and a single electrode 38 positioned near the proximal end 20. These single electrodes are positioned on the axial centerline of the paddle. Significantly, with the present invention, within the first pair width there are three coincident electrode pairs 32a and 34a, 32b and 34b, and 36 and 38 spaced axially along the paddle. As a result of this configuration, all straight nerve fibers passing within the first pair width along the axial length of the paddle will be subject to stimulation from two electrode contacts.

Variations of the preferred embodiments are possible without being outside the scope of the present invention. Therefore, to particularly point out and distinctly claim the subject matter regarded as the invention, the following claims conclude the specification.

What is claimed is:

1. An implantable medical lead for electrical stimulation, comprising:
a lead paddle having a paddle distal end, a paddle proximal end, a paddle first side, a paddle second side, and a paddle axial length, the lead paddle is curved from the paddle first side to the paddle second side to match the shape of a human dura mater;
a first pair of electrodes centrally located on the lead paddle with a first electrode positioned near the periphery of the paddle first side and a second electrode position near the periphery of the paddle second side, a first pair width defined by the distance between the first electrode and the second electrode, wherein the first pair width is free of electrodes;
a second pair of electrodes positioned between the first pair of electrodes and the paddle distal end, the second pair of electrodes having a second pair width that is less than the first pair width, wherein the second pair width is free of electrodes, and wherein the lead paddle is free of electrodes between the first pair of electrodes and the second pair of electrodes;
a third pair of electrodes positioned between the first pair of electrodes and the paddle proximal end, the third pair of electrodes having a third pair width that is less than the first pair width, wherein the third pair width is free of electrodes, and wherein the lead paddle is free of electrodes between the first pair of electrodes and the third pair of electrodes;
a fifth electrode positioned between the second pair of electrodes and the paddle distal end, the fifth electrode positioned between the paddle first side and the paddle second side within the second pair width; and
a sixth electrode positioned between the third pair of electrodes and the paddle proximal end, the sixth electrode positioned between the paddle first side and the paddle second side within the third pair width,
whereby within the first pair width there is at least two coincident electrode pairs spaced axially along the paddle to provide electrical stimulation of nerve fibers at two points along the axial length of the lead paddle.

2. The implantable medical lead as in claim 1, wherein the lead paddle comprises silicone rubber.

3. The implantable medical lead as in claim 1, wherein the first pair of electrodes are the only electrodes positioned axially between the second pair of electrodes and the third pair of electrodes.

4. The implantable medical lead as in claim 1, whereby within the first pair width there is three coincident electrode pairs spaced axially along the paddle to provide electrical stimulation of nerve fibers at two points along the axial length of the lead paddle.
